(19) United States
(12) Patent Application Publication
(10) Pub. No.: US 2011/0105838 A1
(43) Pub. Date: May 5, 2011

(54) SUCTION DEVICE FOR ENDOSCOPIC INSTRUMENTS AND METHOD

(76) Inventor: ROBERTO FOGEL, Miami, FL (US)

(21) Appl. No.: 12/915,944

(22) Filed: Oct. 29, 2010

Related U.S. Application Data
(60) Provisional application No. 61/256,252, filed on Oct. 29, 2009.

(51) Int. Cl. A61B 1/012; A61B 17/94

(52) U.S. Cl. 600/104; 600/156

(57) ABSTRACT

The present invention is related to a suction device especially designed for endoscopic instruments, including at least two cylindrical suction ports located at both sides of the distal end of the endoscopic instrument. Each suction port has inside and next to the distal end thereof at least one sharp traversing blade. Each suction port is connected to a suction line in turn connected to a suction pump. The external end of said suction line is connected to a disposal recipient into which the debris, blood, coagula, etc. removed from the surgical site will be disposed.

### BACKGROUND OF THE INVENTION

1. Field of the Invention

The present invention relates generally to endoscopic instruments, including but not limited to endoscopes, arthroscopes, etc., and more particularly is referred to a suction device applicable to endoscopic instruments through which blood and coagula are immediately sucked from the surgical field without the necessity of taking the instrument out of the endoscope to suck the blood on the surgical field and then re-enter the instrument to continue the procedure. Even more particularly, the present invention is directed to a suction device for endoscopic instruments for removing from the surgical field blood and any other element that is obstructing the surgeon vision during the procedure.

2. Description of the Prior Art

Endoscopy is a minimally invasive diagnostic medical procedure that is used to assess the interior surfaces of an organ by inserting a tube into the body. The instrument may have a rigid or flexible tube and not only provide an image for visual inspection and photography, but also enable taking biopsies and retrieval of foreign objects. Endoscopy is the vehicle for minimally invasive surgery, and patients may receive conscious sedation so they do not have to be consciously aware of the discomfort.

Many endoscopic procedures are considered to be relatively painless and, at worst, associated with moderate discomfort; in esophagogastroduodenoscopy, for example, most patients tolerate the procedure with only topical anesthesia of the oropharynx. Complications are rare but can include perforation of the organ under inspection with the endoscope or biopsy instrument. If that occurs, open surgery may be required to repair the injury.

The procedure is performed using an endoscope which basically comprises a rigid or flexible tube, a light delivery system to illuminate the organ or object under inspection. The light source is normally outside the body, and the light is typically directed via an optical fiber system, and a lens system transmitting the image to the viewer from the fiberscope. It usually includes an additional channel to allow entry of medical instruments or manipulators, including scissors, staplers, etc.

This technology is being used successfully during the last years for performing modern and revolutionary gastric procedures, including all types of bariatric procedures. During these procedures the surgeon must enter the stomach through the patient's mouth and esophagus and once the portion or the organ is reached, different type of instruments may be inserted through the endoscope for performing the procedure.

One of the situations the surgeon most commonly must face during the procedures is removing blood, coagula, and debris from the surgical field. In order to be able to do that, some kind of suction apparatus should be inserted for which the other instruments should be taken out. The main object of the present invention is to avoid this situation in the future, by providing to the endoscopic instruments with an appropriate suction device capable of sucking blood, coagula, and any type of debris while the procedure is being performed.

There are several instruments and devices for performing surgical endoscopic procedures including suction lines. For example, U.S. Pat. No. 5,626,560 describes a diathermic hand-held instrument and a detachable endoscopic probe for use in surgical operations, which provides for both coagulation and dissection in one tool. In particular, a sonotrode that receives a gas supply line and a suction line is embodied to be electrically conductive. The endoscopic probe also may be attached to alternative hand-held instruments.

U.S. Pat. No. 5,605,537 describes a multifunctional device for use in endoscopic medical procedures, capable of suction, irrigation and aqua-dissection, comprising an elongated tube that is open at both ends, a pair of valves in communication with the tube such that the device may be used for either suction, irrigation, or aqua-dissection, and further having an optional shield which may be attached for the suctioning of large volumes of fluid therethrough. A removable seal is attached to the proximal end of the tube, which seal can be replaced with an optional end cap for allowing the tube to be used to transmit other instruments into the operative field, which other instruments can be used simultaneously with the device while the device is performing suction or irrigation.

Another example is the U.S. Pat. No. 5,607,391 which comprises an endoscopic surgical instrument for aspiration and irrigation of a surgical site. Connection ports for irrigation fluid and a suction source are provided, which communicate with a single lumen cannula which transports both the irrigation fluid and the suction pressure to the surgical site. A valve mechanism having a novel valve stem and valve arrangement is also disclosed for providing higher pressure capabilities for the instrument. In one embodiment, the single lumen cannula is provided with a sliding sleeve to vary the pressure of the irrigation fluid to provide for high pressure application of the irrigation fluid to perform hydrodissection. The aspiration-irrigation instrument may also include electrocautery capabilities and a detachable cannula assembly for interchanging cannulas having different working tool members.

U.S. Pat. No. 5,624,453 describes a flexible endoscopic instrument with a plurality of elastic ligating rings mounted on one tube of a pair of tubular members which are affixed in coaxial relation to the insertion end of an endoscope. The endoscope is equipped with illumination and viewing means to facilitate orientation of the instrument in the body organ, and longitudinally extending tubular passages comprising a channel through which objects may be passed and suction applied for drawing the lesion tissue into the tubular end of the endoscope to facilitate ligation of a lesion, and a working channel through which a flexible actuating cable is inserted. Each of the elastic rings can be dislodged from the endoscope and placed in ligating relation to a lesion when lesion tissue is drawn into the innermost of the tubular members by a suction force applied through the suction channel and each of the rings can be applied to a different one of the multiple lesions in the body organ during a single insertion of the endoscope.

Also U.S. Pat. No. 5,665,100 describes an endoscopic instrument, including a forceps unit for being positioned within an anatomical cavity and a removable operating unit. The operating unit includes a hub mounting an inner tubular member removably disposed at least partly within the intermediate member and carrying operating members for performing at least one of the functions of cutting, grasping, manipulating, dissecting, collecting tissue for biopsy, penetrating tissue with a needle, injecting fluids, creating suction, aspirating, irrigating, suturing, ligating, visualizing, illuminating and cauterizing.

U.S. Pat. No. 5,667,475 shows an instrument for endoscopic subfascial discission of perforans veins (ESDP) consists of a surgical endoscope and a tube. The distal end of the tube extends beyond the distal end of be surgical endoscope and is designed as a thicker, atraumatic lip that contains a suction channel diametrically arranged to a front lens system and connected through a space between the shaft and the tube to a connection fitting for a suction hose.

Furthermore, U.S. Pat. No. 5,562,640 describes an endoscopic surgical instrument for aspiration and irrigation of a surgical site. The device includes at least one rotatable trumpet valve to provide for variable orientation of the device during use. Connection ports for irrigation fluid and a suction means are provided which communicate with a single lumen cannula which transports both the irrigation fluid and the suction pressure to the surgical site. The single lumen cannula is provided with a sleeve means to vary the pressure of the irrigation fluid to provide for high pressure application of the irrigation fluid to perform hydrodissection. A plurality of dissector tips and a novel means of securing the tips to the single lumen cannula are also disclosed.

Finally, U.S. Pat. No. 5,755,724 teaches about a collection system for collecting substances within the body in endoscopic operative procedures wherein access to a site in the body is obtained through a narrow portal in the body includes an elongate inner tubular member, an elongate outer tubular member receiving the inner tubular member, a collection bag carried by a distal end of the inner tubular member and a suction cutter disposed in the inner tubular member. The collection bag has a non-expanded position wherein the collection bag is disposed within the outer tubular member to facilitate the introduction of distal ends of the outer and inner tubular members through the narrow portal. The collection bag is movable from the non-expanded position to an expanded position within the body, the collection bag in the expanded position being disposed externally of the outer tubular member to allow substances to be introduced in the collection bag. The suction cutter has a distal end disposed within the collection bag, a proximal end and a suction channel between the distal and proximal ends of the suction cutter. The suction channel is connectible with a source of suction externally of the body to withdraw the substances introduced in the collection bag while the collection bag remains in the body.

Even though the above cited methods address some of the needs of the market, a more effective suction device capable of providing a blood removal effect on the surgical site while the surgical procedure is being performed is still desired.

### SUMMARY OF THE INVENTION

This invention is directed to a novel suction device especially designed for endoscopic instruments, including at least two cylindrical suction ports located at both sides of the distal end of the endoscopic instrument, each having a sharp traversing blade.

In one general aspect of the present invention, the suction device comprises two suction ports connected to respective suction lines in turn connected to a suction pump for delivering blood, coagula and debris to a disposal recipient.

Another aspect of the present invention provides a suction device comprising four suction ports surrounding the distal end of the endoscopic instrument, each of which having a traverse sharp edge and connected to a suction line.

Some of the advantage of the present invention may be summarized as:

It creates an even suction effect on the surgical field;

It allows the removal not only of blood but also solid debris and coagula without risk of blocking the suction line;

It allows the destruction of any coagula or debris by cutting them with the sharp blade incorporated into the suction port;

It avoids the cumbersome procedure of taking the instruments out of the patient for inserting the suction device for removing blood and coagula from the surgical field.

In summary, the present invention is related to a suction device especially designed for endoscopic instruments, including at least two cylindrical suction ports located at both sides of the distal end of the endoscopic instrument. Each suction port has inside and next to the distal end thereof at least one sharp traversing blade. Each suction port is connected to a suction line in turn connected to a suction pump. The external end of said suction line is connected to a disposal recipient into which the debris, blood, coagula etc removed from the surgical site will be disposed.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a general schematic perspective view of an endoscopic biopsy forceps, including the suction device in accordance with the present invention.

FIG. 2 is another perspective view of the endoscopic instrument showing the moment in which the forceps is open by moving backwards the mechanical command. In this figure, it could also be seen the suction line coming from the central lumen of the instrument.

FIG. 3 is another perspective view showing the moment in which the forceps is open and is approaching a stomach tissue.

FIG. 4 is another perspective view showing how the forceps command is moving forward closing the forceps jaws.

FIG. 5 is another perspective view showing the forceps grabbing the tissue, blood flowing from the tissue and being sucked by the suction ports and then blood and debris going through the suction line.

FIG. 6 is similar to FIG. 5 but this time illustrating blood going through the suction line towards the disposal bag.

FIG. 7 is a frontal perspective view showing the forceps grabbing the stomach tissue, and blood being removed from the surgical site by the suction ports and then going through the suction line.

FIG. 8 shows schematically the sucked blood going through the suction line and entering into the disposal recipient.

FIG. 9 is a perspective view of a second embodiment in which the endoscopic instrument is now an endoscopic scissor. It shows the moment in which the scissor is open by moving backwards the mechanical command. In this figure, it could also be seen the suction line coming from the central lumen of the instrument.

FIG. 10 is another perspective view showing the moment in which the endoscopic scissor is open and is approaching a stomach tissue.

FIG. 11 is another perspective view showing how the scissor command is moving forward closing the scissor blades.

FIG. 12 is a frontal perspective view showing the scissor cutting the stomach tissue, and blood flowing and being removed from the surgical site by the suction ports and then going through the suction line.

FIG. 13 shows schematically how sucked blood goes through the suction line towards the disposal recipient; finally:

FIG. 14 shows schematically the sucked blood going through the suction line and entering into the disposal recipient.

### DETAILED DESCRIPTION OF REPRESENTATIVE EMBODIMENTS

Shown throughout the Figures, the invention is directed to a suction device 10 for endoscopic instruments 100, including at least two cylindrical suction ports 11 located at both sides of the distal end 101 of the endoscopic instrument 100.

Each suction port 11 is defined by a cylindrical tube located one at each side of the distal end 101 of the endoscopic instrument 100. Each tube 11 has inside and next to the distal end thereof at least one sharp traversing blade 12. This blade has the very important mission of cutting any debris or coagulum the suction port may remove from the surgical site.

Furthermore, each suction port 11 is connected to a suction line 13 in turn connected to a suction pump (not illustrated). The external end 14 of said suction line 13 is connected to a disposal recipient 120 into which debris, blood, coagula, etc. removed from the surgical site will be disposed.

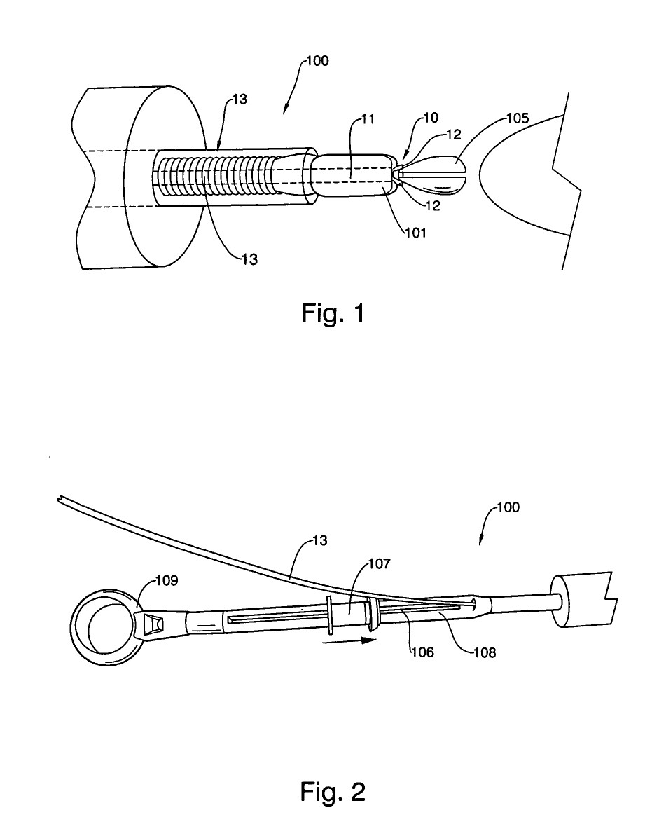

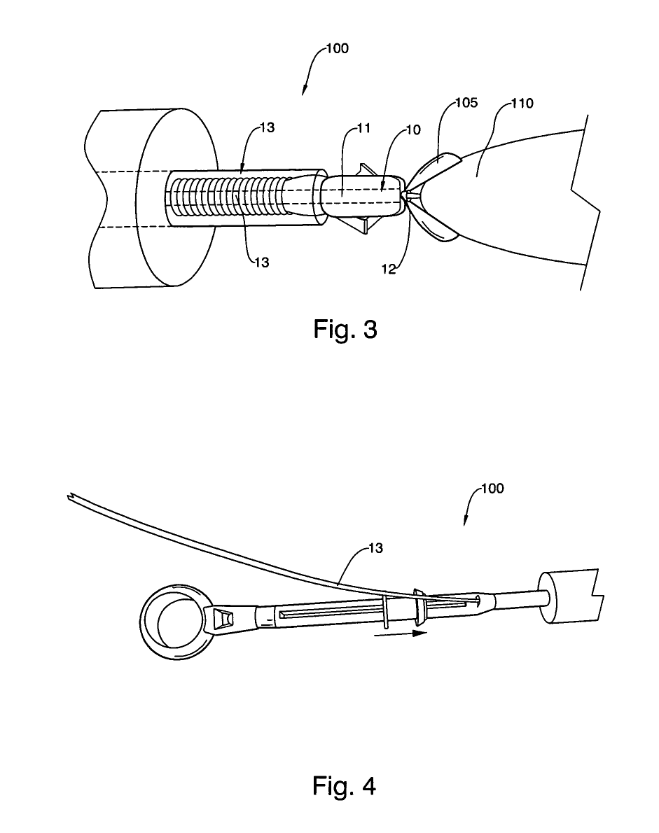

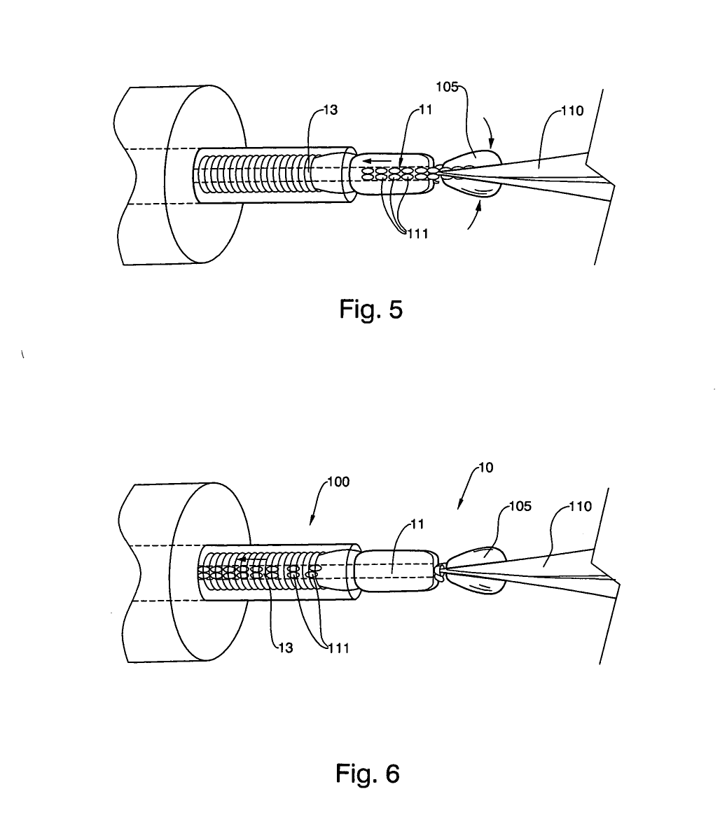

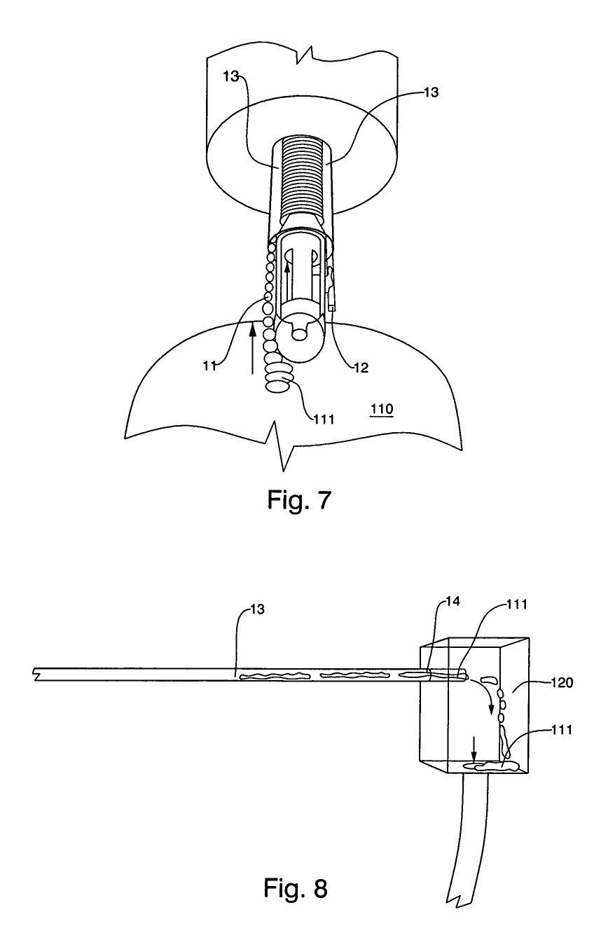

FIGS. 1-8 illustrate a first embodiment of the present invention in which the endoscopic instrument 100 is an endoscopic biopsy forceps. As usual, said instrument includes at its distal end two movable grabbing jaws 105 pivotally connected to the distal end 101. Said jaws are connected through a commanding cable 106 to a control device 107. In this embodiment, this control device comprises a mechanical piston linearly movable on longitudinal tracks 108. A handle 109 at the external end allows the surgeon to manipulate it easily.

When the above mentioned piston 107 is moved backwards (FIG. 2) said cable 108 is pulled and jaws 105 are open. When said piston is moved forward, jaws 105 are closed. This mechanism is not disclosed in detail as is not part of the present invention.

The endoscopic procedure starts when instrument 100, such as an endoscopic biopsy forceps, is introduced into the patient's stomach transorally. The control command 107 is moved backwards to open the forceps jaws 105. Then forceps 100 is approached to the stomach section 110 to be studied. The suction pump (not illustrated) is activated to create a suction effect of both suction ports 11. Thus, a suction effect is created around the forceps grabbing the stomach tissue 110. Blood 111 is removed through the suction ports 11 while the forceps grabs tissue 110. Blood 111, coagula and debris sucked by the suction port 11 from the surgical sites are then transferred by this suction effect to the suction line 13 towards a disposal bag 120.

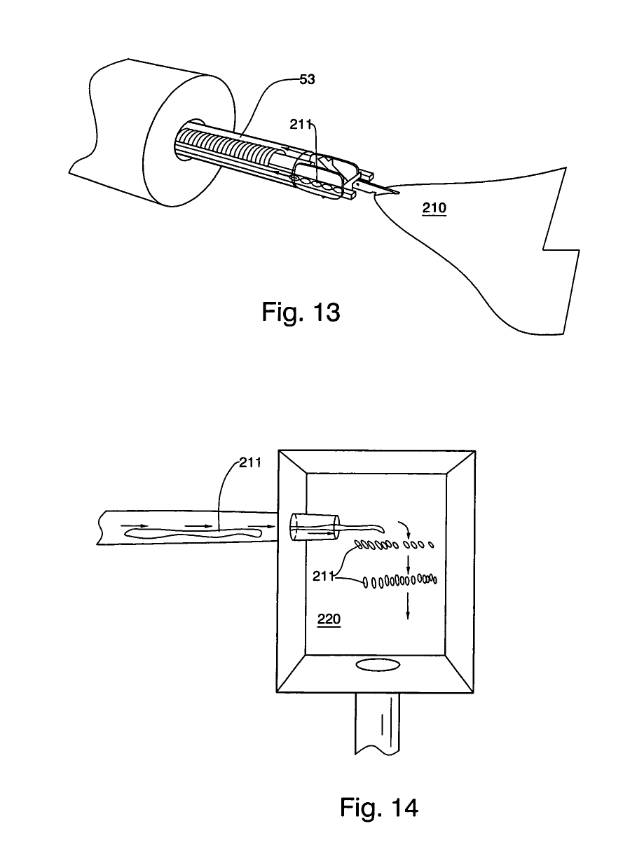

FIGS. 10, 12 and 13 illustrates a second embodiment of the present invention, comprising this time a suction device 50 for an endoscopic scissor 200, including at least two cylindrical suction ports 51 located at both sides of the distal end 201 of the endoscopic scissor 200.

Each suction port 51 is defined by a cylindrical tube located one at each side of the distal end 201 of the endoscopic instrument 200. Each tube 51 has inside and next to the distal end thereof at least one sharp traversing blade 52. This blade has the very important mission of cutting any debris or coagulum the suction port may remove from the surgical site.

Furthermore, each suction port 51 is connected to a suction line 53 in turn connected to a suction pump (not illustrated). The external end 54 of said suction line 53 is connected to a disposal recipient 220 into which debris, blood, coagula, etc. removed from the surgical site will be disposed.

As usual said endoscopic scissor includes at its distal end two movable blades 205 pivotally connected to the distal end 201. Said blades are connected through a commanding cable 206 to a control device 207. In this embodiment this control device 207 comprises a mechanical piston linearly movable on longitudinal tracks 208. A handle 209 at the external end allows the surgeon to manipulate it easily.

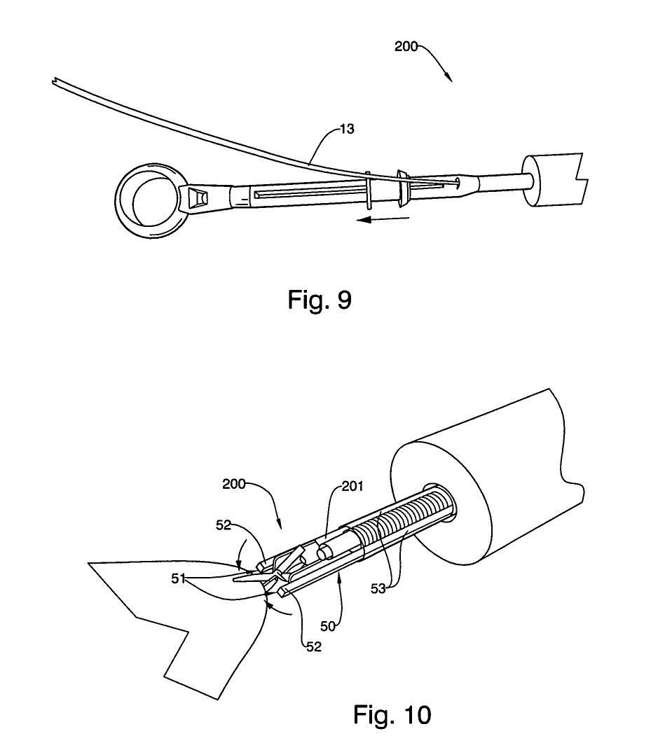

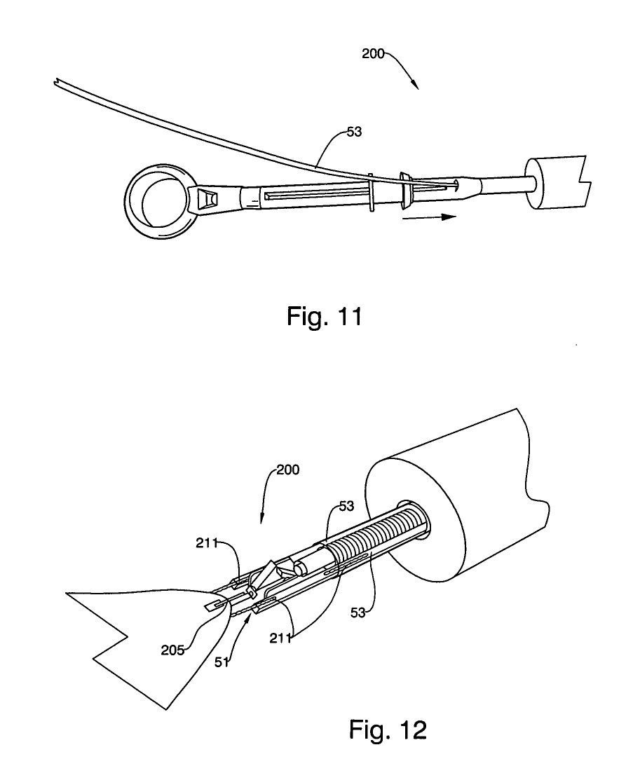

When the above mentioned piston 207 is moved backwards (FIG. 9) said cable 208 is pulled and blades 205 are open. When said piston is moved forward (FIG. 11), blades 205 are closed and the tissue is cut. This mechanism is not disclosed in detail as is not part of the present invention.

The endoscopic procedure starts when instrument 200, such as an endoscopic scissor, is introduced into the patient's stomach transorally. The control command 207 is moved backwards to open the scissor blades 205. Then scissor 200 is approached to the stomach section 210 to be studied. The suction pump (not illustrated) is activated so as to create a suction effect of both suction ports 51. Thus, a suction effect is created around the scissor blades cutting the stomach tissue 210. Blood 211 is removed through suction ports 51 while the scissor cuts tissue 210. Blood 211 and/or coagula and/or debris sucked by the suction port 51 from the surgical site are then transferred by this suction effect to the suction line 53 towards a disposal bag 220.

Even though in the present specification only two instruments have been described, this should not be considered a limitation of the present invention, as any other instruments used during surgical procedures of this type may be used, namely: clips, lups, injectors, polypectomy snare, ERCP catheters, graspers.

While the preferred embodiments of the invention have been described above, it will be recognized and understood that various modifications can be made in the invention and the appended claims are intended to cover all such modifications which may fall within the spirit and scope of the invention.

I claim:

1. Suction device for endoscopic instruments, including suction ports located at the distal end of the endoscopic instrument; said suction port includes at least one sharp traversing blade; and each suction port is connected to a suction line in turn connected to a suction pump; the external end of said suction line is connected to a disposal recipient.

2. The suction device for endoscopic instruments, in accordance with claim 1, wherein the instrument includes at least two cylindrical suction ports.

3. The suction device for endoscopic instruments, in accordance with claim 2, wherein said suction ports are located at both sides of the distal end of the instrument.

4. The suction device for endoscopic instruments, in accordance with claim 1, wherein the instrument is an endoscopic biopsy forceps.

5. The suction device for endoscopic instruments, in accordance with claim 1, wherein the instrument is an endoscopic scissor

6. The suction device for endoscopic instruments, in accordance with claim 1, wherein the device has four suction ports surrounding the distal end of the endoscopic instrument.

7. The suction device for endoscopic instruments, in accordance with claim 1, wherein into said disposal recipient debris, blood, coagula removed from the surgical site are disposed.

8. The suction device for endoscopic instruments, in accordance with claim 1, wherein inside each suction port a diametrically disposed sharp blade defines a cutting means inside said port capable of cutting any debris, coagula or the like removed from the surgical field during the endoscopic procedure.

9. The suction device for endoscopic instruments, in accordance with claim 1, wherein said ports are connected to two independent parallel suction tubes which are part of the suction line.

10. The suction device for endoscopic instruments, in accordance with claim 1, wherein the suction lines are connected to a disposal bag.

11. The suction device for endoscopic instruments, in accordance with claim 10, wherein the disposal bag is especially designed for containing and disposing biological material.

12. The suction device for endoscopic instruments, in accordance with claim 4, wherein the endoscopic biopsy forceps includes at its distal end two movable grabbing jaws pivotally connected to the distal end, being said jaws connected through a commanding cable to a control device.

13. The suction device for endoscopic instruments, in accordance with claim 12, wherein said control device comprises a mechanical piston linearly movable on longitudinal tracks and a manipulation handle.

14. The suction device for endoscopic instruments, in accordance with claim 5, wherein said endoscopic scissor includes at its distal end two movable blades pivotally connected to the distal end, and said blades are connected through a commanding cable to a control device.

15. The suction device for endoscopic instruments, in accordance with claim 14, wherein said control device comprises a mechanical piston linearly movable on longitudinal tracks, and a manipulation handle.

16. The suction device for endoscopic instruments, in accordance with claim 1, wherein the instrument is one of the following: clips, lups, injectors, polypectomy snare, ERCP catheters, graspers.

17. The suction device for endoscopic instruments, in accordance with claim 1, wherein the instrument is an endoscopic scissor

18. A suction method for endoscopic procedures, comprising the steps of:
a) introducing an endoscopic instrument into the patient's stomach transorally, including said instrument suction ports at its distal end;
b) activating the control command located at the instrument external end to open the instrument;
c) approaching the instrument to the stomach section to be studied;
d) activating a suction pump connected to the suction ports;
e) creating a suction effect in the suction ports around the instrument grabbing the stomach tissue;
f) removing blood through the suction ports while the instrument grabs the tissue;
g) transferring blood, coagula and debris sucked by the suction port from the surgical site are to a disposal bag.

19. The method of claim 18, wherein the instrument is an endoscopic biopsy forceps.

20. The method of claim 18, wherein the instrument is one of the following: scissors, clips, lups, injectors, polypectomy snare, ERCP catheters, graspers.
